(19) United States
(12) Patent Application Publication
(10) Pub. No.: US 2013/0033968 A1
(43) Pub. Date: Feb. 7, 2013

(54) Sparker Array Source

(71) Applicants: Raymond B. Schaefer, Lexington, MA (US); Michael Grapperhaus, Dracut, MA (US); John Gallagher, Goffstown, NH (US); Robin O. Cleveland, Jamaica Plain, MA (US)

(72) Inventors: Raymond B. Schaefer, Lexington, MA (US); Michael Grapperhaus, Dracut, MA (US); John Gallagher, Goffstown, NH (US); Robin O. Cleveland, Jamaica Plain, MA (US)

(21) Appl. No.: 13/515,453

(22) PCT Filed: Dec. 15, 2010
(86) PCT No.: PCT/US10/60503

§ 371 Date: Jun. 12, 2012

Related U.S. Application Data
(60) Provisional application No. 61/289,125, filed on Dec. 22, 2009.

(51) Int. Cl. A61B 18/26; A61N 5/00

(52) U.S. Cl. 367/140

(57) ABSTRACT

A sparker array includes a plurality of sparker sources of sound and light emissions, the plurality of sparker sources arranged in a geometric pattern with respect to a region, the array configured to deliver a maximal acoustic output to the region. Sparker sources may include reflectors. A single electrical source to drive a sparker array may be employed. A sparker system may include two or more sparker arrays. A time delay may be employed to trigger electrical circuits of the sparker arrays. Sparker arrays may be used to deliver shock waves with increased operational life, consistency and efficacy for specific applications.

### RELATED APPLICATION

This application claims the benefit of U.S. Provisional Application No. 61/289,125, filed on Dec. 22, 2009. The entire teachings of the above application is incorporated herein by reference.

### GOVERNMENT SUPPORT

This invention was supported in part in by the National Institutes of Health (NIH) Small Business Innovation Research (SBIR) program under Grant #2R44DK074231-02 and Grant #1R43DK089703-01. The Government has certain rights in the invention.

### BACKGROUND

Sparkers are pulsed sound and light sources known in the art which employ pulsed electric discharges in a liquid to generate high pressure shock waves and, simultaneously or separately, light emissions. A wide range of sizes and designs are used for various applications. For instance, a lithotripter comprising a single sparker placed at one focus of a semi-ellipsoid reflector is used to generate a shock wave which breaks up kidney stones located at the second focus (see FIG. 1). This is a non-invasive extra-corporeal method for treating kidney stones. In another application, a single sparker placed at the focus of a paraboloid reflector is used to generate a shock wave that is directed into a pipe or at an area to control zebra mussels, an invasive species that clogs water supplies.

In many sparkers known in the art, the pulsed electric discharge is generated between two electrodes separated by a gap. The output and/or life of this type of sparker are limited by electrode erosion, which may increase the gap separation, rendering the source output insufficient. For instance, conventional sparkers used in lithotripsy must be replaced during a medical procedure, since 2,000-3,000 pulses are required to break up the stone sufficiently, whereas the life of the sparker is about 1,500 pulses. This need for sparker replacement is an inconvenience to the procedure. Furthermore, the spark that generates the shock wave arises from a pulsed electric discharge jumping from one electrode to another, which “wanders” from pulse to pulse. The result is that the shock may not be generated precisely at the focus, causing imprecise focusing at the second focus which, in lithotripsy, is a likely contributor to unpleasant side effects in patients. This type of effect may be deleterious in any application in which the shock is generated at a focal region and transferred to another region.

Also, in conventional shock wave lithotripters, the position of a kidney stone is not detected continuously during a procedure. Consequently, due to movement of the stone from the shock or from breathing by the patient, shocks may miss or only partially hit the stone. This increases the number of shocks needed to break up the stone and increases side effects from the shock hitting tissue in the vicinity of the stone.

In addition, pressure pulses from conventional single sparkers have been use to tenderize meat, poultry and fish. Because of spreading of the pressure pulse, the tenderization effect may be non-uniform.

### SUMMARY

The present invention relates to a sparker array or multiple sparker arrays. Each sparker in the array can be a pulsed sound and/or light source. While embodiments of the invention are described primarily with respect to sound emissions and resulting pressure profiles, it will be understood that the same inventive concepts apply to light emissions and light intensity profiles.

Each sparker array may be driven with a single pulsed power circuit. For a given pulsed power circuit there may be an optimum number of sparkers for maximizing efficiency and lifetime. The array is arranged so that the shock waves from each sparker element arrive at a specific location or region to provide a desired combined pressure profile in space and time. Embodiments of the invention may include the addition of acoustic reflectors, designed to deliver a desired combined pressure profile in space and time, the implementation of which may range from having a reflector for each sparker element of each array to having a single reflector for the entire array. Each reflector may be an ellipsoidal reflector having a first and second focus. Multiple reflectors may be positioned so that all of their second foci are at the same location, where, for instance, a kidney stone could be located in a medical procedure. Each sparker in the array may have an electrode positioned at the first focus of an ellipsoidal reflector.

In another embodiment, each reflector may be a paraboloid, with the sparker placed at the focus. Alternatively, the entire array may have a single reflector to increase the efficiency of utilizing the omnidirectional pressure from the sparkers in the array. In general, the array may be arranged to deliver pressure to a region or planar surface where, for instance, meat, fish, poultry or the like may be located for the purpose of tenderization and/or disinfection. For applications such as tenderization, the sparker array can provide a more uniform pressure pulse than a single sparker. In general, both the light and pressure pulse emitted from the sparker array may work together to effect a particular result in a target region.

The sound and light emissions may occur in a liquid, such as a coupling liquid. The salinity of the liquid may be greater than 1 millisiemens per centimeter. Because of the salinity of the liquid, each sparker can have a single electrode, with the liquid acting as the second electrode.

Also, the geometrical arrangement of the sparkers may be selected in order to deliver a desired pressure distribution to a specific region. In addition, the sound and light emissions can include pulses having an adjustable temporal pulselength and the pulselength may be adjusted to adjust the pressure profile delivered to the region. For example, the temporal pulselength of the sound emission may be adjusted to adjust the size of the focal region. Furthermore, in instances in which the sparker electrode is in the vicinity of an electrically conducting material, e.g., as in the case of a metal reflector, the sparker design may include an electrical insulator to assure proper operation and long lifetime. For multiple array arrangements, embodiments of the present invention may include configuring firing of the arrays simultaneously to increase the pressure delivery to a region. Alternatively, embodiments of the present invention may include the capability to trigger shock waves from each array with controlled time separations. For example, two sparker arrays may be triggered to deliver one pulse each, the pulses separated by a time interval. Sparker arrays used in a two-pulse or multi-pulse mode, in which the time between pulses may range over 1-1000 microseconds, may be more efficient at breaking up kidney stones. Furthermore, the sparker array or multiple sparker arrays may be arranged to allow for observation of a specified region. For example, the array elements, including any reflectors, may be arranged to allow for observation of the second focus (of all the array elements), so that a sensor can track the position of a kidney stone at that focus.

Embodiments of the present inventive sparker array can be used to improve consistency and efficiency, while increasing the useful life of the sparker system. The inventive improvement increases the capability of the source and/or reduces the requirements' on the emissive source to accomplish an intended objective. For lithotripsy, the sparker array can increase the life of the sparker, reduce side effects in the patient, provide for using two or more pulses to break kidney stones, and allow for tracking the position of kidney stones during a medical procedure.

Furthermore, it is thought that the size of the focal region is an important feature in achieving comminution, or pulverization, of kidney stones.

Embodiments of the present invention are amenable for use in a wide variety of industrial, commercial, military, academic, and environmental applications such as, in addition to lithotripsy and meat tenderization, surface treatment (e.g. cleaning, barnacle removal), protection against unfriendly divers, sterilization, geophysical exploration, anti-biofouling, underwater surveillance, ballast water control, mine sweeping, submarine countermeasures, controlling zebra mussels and the like.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 illustrates a conventional single sparker lithotripter.

FIG. 2 illustrates an embodiment of a sparker array lithotripter.

FIG. 3 is a schematic diagram of an array of sparkers arranged to provide a specific pressure level to a specific region including a single electrical circuit to drive all the sparkers in the array.

FIG. 4 illustrates different optimal number of sparkers for different pulsed power circuits.

FIG. 5 illustrates an array in which each sparker includes a reflector, increasing the pressure level delivered to a specific region.

FIG. 6 illustrates an array in which each sparker includes a semi-ellipsoidal reflector, with an electrode at one focus, and the array arranged so that all the reflectors have the same second focus.

FIG. 7 illustrates an array with a single reflector for the entire array.

FIGS. 8A-8C show examples of different geometrical configurations for the elements of the array.

FIG. 9 illustrates an example configuration for a sparker with an electrically conducting reflector.

FIG. 10 shows a configuration of two sparker arrays, each of which can be triggered electronically at different times.

FIG. 11 illustrates an array arrangement including a sensor for observing the region with the maximum pressure level.

FIG. 12 illustrates an example pulsed electrical driver circuit for a single sparker array.

FIGS. 13A and 13B show exemplary simulation data from a model of a sparker array.

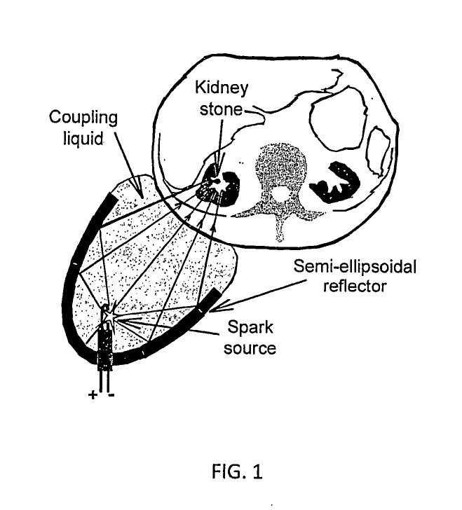

### DETAILED DESCRIPTION

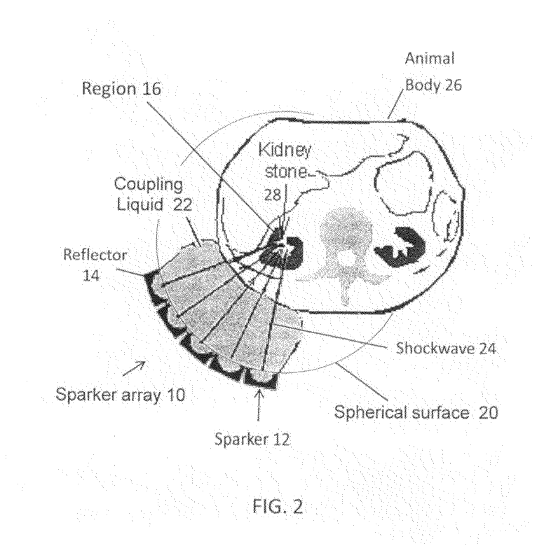

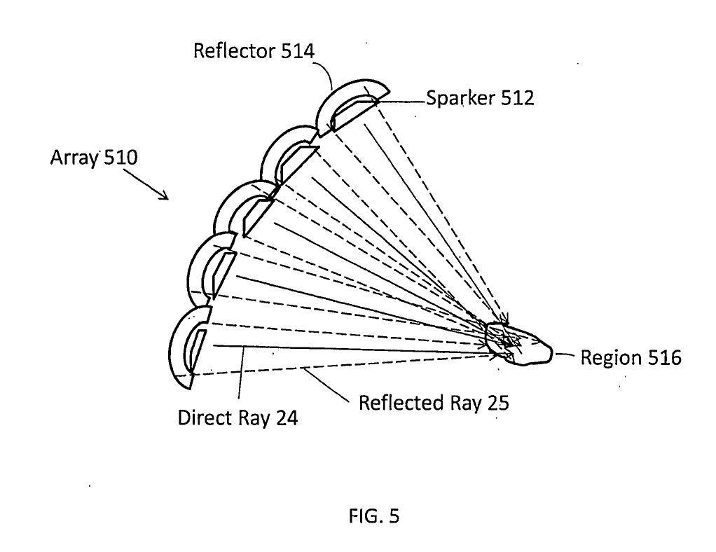

Shown in FIG. 2 is an embodiment of a sparker array lithotripter. Sparker array 10 includes multiple sparkers 12 arranged in a geometric pattern to provide a maximum pressure level in specific region 16. In a preferred embodiment, the geometric pattern may be the surface of a sphere 20. The center of the sphere of spherical surface 20 may lie within specific region 16. Furthermore, sparkers 12 may include reflectors 14, which may be ellipsoid reflectors. Ellipsoid reflectors 14 may be arranged on spherical surface 20 with the second foci of all ellipsoid reflectors 14 located at the center of the sphere. In a preferred embodiment, sparkers 12 may generate pulsed electrical discharges in coupling liquid 22 to generate high pressure shock waves 24. Shock waves 24 may comprise direct and reflected shock waves, or rays, as described below with reference to FIG. 5. Only direct show waves are illustrated in FIG. 2. The salinity of liquid 22, which is a measure of the conductivity of the liquid, may be greater than 1 millisiemens/cm. When used as a lithotripter, an embodiment of sparker array 10 may be placed adjacent to the animal body 26 such that region 16 is located within the body 26. Region 16 located in body 26 may include a kidney stone 28. In a preferred embodiment, shock waves 24 from each sparker 12 arrive at specific location 16 to provide a desired combined pressure profile in space and time for the treatment of kidney stone 28.

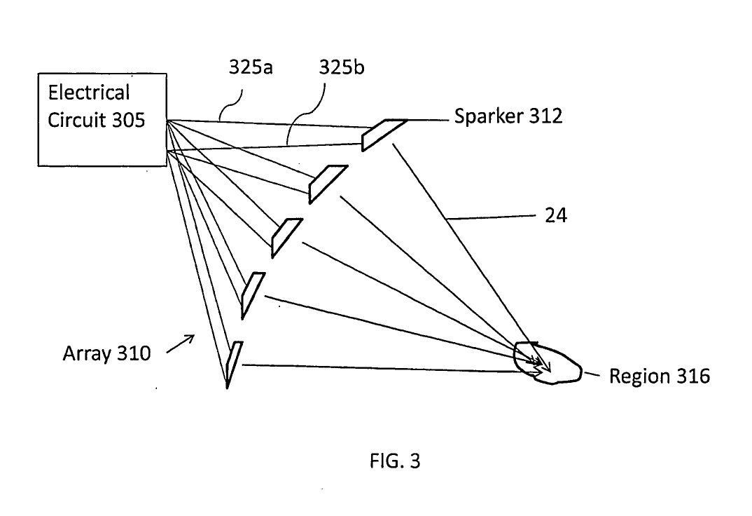

Shown in FIG. 3 is a schematic diagram of an embodiment of sparker array 310 having sparkers 312, positioned so that a specific pressure is produced in specific region 316. Any number of sparkers 312 can be included in array 310 and their positions selected to produce a variety of maximal regions. For simplicity, the sound emitted from each sparker 312 is illustrated as a single and direct shock wave 24 from each sparker 312 to region 316.

As shown in FIG. 3, a single electrical circuit 305 may drive all of the sparkers 312 in array 1. As illustrated, all sparkers 312 are connected in parallel to circuit 305 via connections 325a and 325b. Connections 325a and 325b illustrate a completed circuit. For example, either connection 325a or connection 325b can be a positive lead with the other a negative lead. In general, circuit 305 may have a capacitance of between 0.002 and 128 microfarads (uF), and the charge voltage may be between 3 and 32 kilovolts (kV). In an embodiment, circuit 305 may have a capacitance of between 0.002 uF and 0.64 uF, and the charge voltage may be between 16 and 32 kilovolts (kV). In another embodiment, the capacitance may be between 32 uF and 128 uF with a charge voltage between 3 kV and 12 kV. Other embodiments may feature different capacitance and charge voltage values. Sparkers 312 or array 310 may be positioned to produce a maximum pressure in specific region 316. For simplicity, the sound emitted from each sparker 312 is illustrated as a single and direct shock wave 24 from each sparker 312 to region 316.

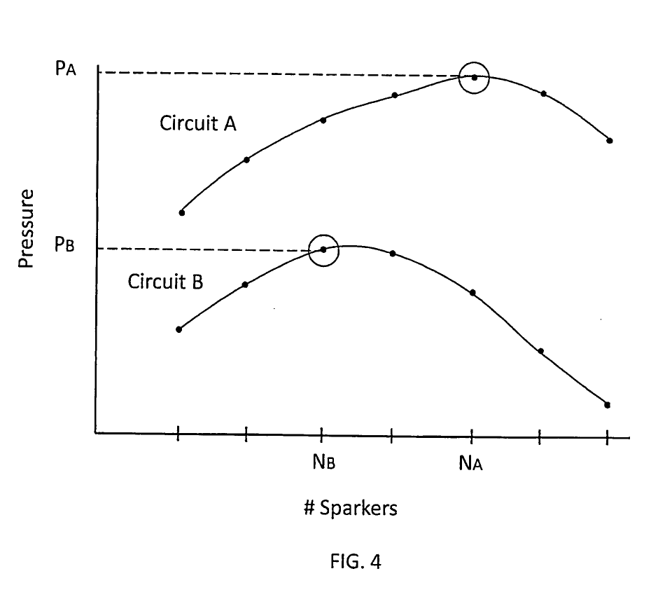

Shown in FIG. 4 is a graph of the total pressure output as a function of the number of sparkers in a sparker array, for two different pulsed power circuits. In each circuit A and circuit B, the same sparker design is used. As shown in FIG. 4, the pressure output, shown on the vertical axis, changes as a function of the number of sparkers driven by each circuit, shown on the horizontal axis. First, the pressure output increases as the number of sparkers driven by each circuit increases, but only up to a point, after which the pressure output decreases as a function of increased number of sparkers. The point at which the pressure output reaches an optimal value is different for circuit A than for circuit B. In FIG. 4, the optimal points for circuits A and B are characterized by number of sparkers N_{A} and N_{B} and pressure outputs P_{A} and P_{B}, respectively. The graph shown in FIG. 4 illustrates the principle that for optimal maximum pressure, the number of sparkers and the pulsed power circuit must be electrically matched. For a given circuit, too few or too many sparkers may result in the circuit not being well matched to the sparker array.

In the embodiment shown in FIG. 5, each sparker 512 of array 510 has a reflector 514 to provide directionality of the sound emission, which, along with the positions of the sparkers in array 1, provides a maximum pressure in a specific region 3. Both direct rays 24 and reflected rays 25 are shown in FIG. 5. The term ray as used herein refers to a ray of light as well as a shock wave or pressure pulse travelling in a specific direction. The direct rays 24 from each sparker travel a different distance from the reflected rays 25, which travel to the reflector first and then are reflected. Depending on the temporal length of the pulse and the extra distance the reflected pulse travels, the pulse profile in region 516 may have a single pulse consisting of an overlap of the direct 24 and reflected 25 rays, or have two pulses, one from the direct rays 24 and the other from the reflected rays 25. Suitable materials for reflectors include air, metal, and plastics, such as Teflon® polytetrafluoroethylene (PTFE) and Delrin® polyoxymethylene (POM), which are highly reflective to sound and light. Teachings of reflectors and reflector systems are disclosed in U.S. Pat. No. 6,672,729 and U.S. Pat. No. 7,593,289, incorporated herein by reference in their entirety.

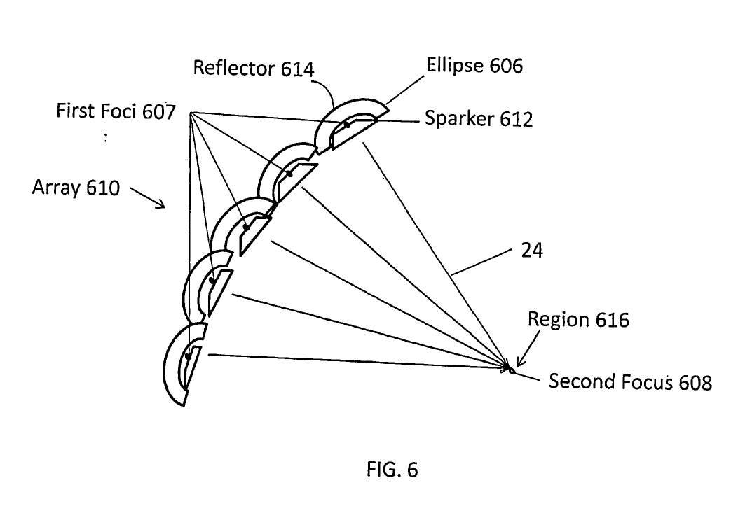

Shown in FIG. 6 is an embodiment in which each sparker source 612 of array 610 includes a reflector 614. Each reflector 614 is positioned to form a section of an ellipse 606, and each sparker 612 is located at the first focus 607 of associated reflector 614. The second focus 608 of each reflector is positioned to provide a maximum pressure in specific region 616. In one preferred embodiment, second focus 608 of each reflector may be at the same location so as to provide a maximum pressure in the region of all the second foci 608. For an ellipsoidal reflector 614, the reflected energy, e.g., the energy of the reflected shock wave, is the major portion of the energy delivered to region 616. For simplicity, the sound emitted from each sparker 612 is illustrated as a single and direct shock wave 24 from each sparker 612 to focus 8 located in region 616.

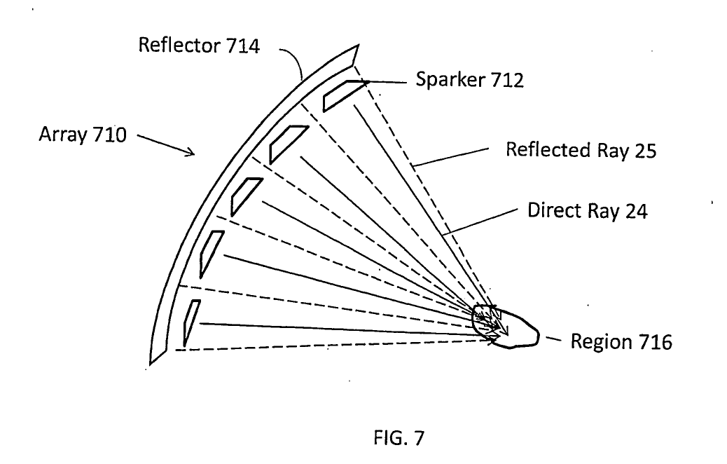

Shown in FIG. 7 is a sparker array 710 with a single reflector 714 that encompasses the array. Both direct rays 24 and reflected rays 25 from the sparker array arrive at region 716 to provide a combined pressure profile in space and time. The shape of the reflector 714 is shown in FIG. 7 to follow the geometric arrangement of sparkers 712, but that need not be the case. As shown in FIG. 7, the geometric arrangement of sparkers 712 in array 710 is such that sparkers 712 are located on a curved line, such as a section of a circle whose center is located in region 716. Other geometric arrangements of the sparkers 712 of array 710 are possible. In general, the geometric arrangement of the sparkers of array 710 may be one dimensional, e.g., along a straight line, two-dimensional, e.g., along a curve, arc, or in a plane, or three-dimensional, e.g., on a spherical surface.

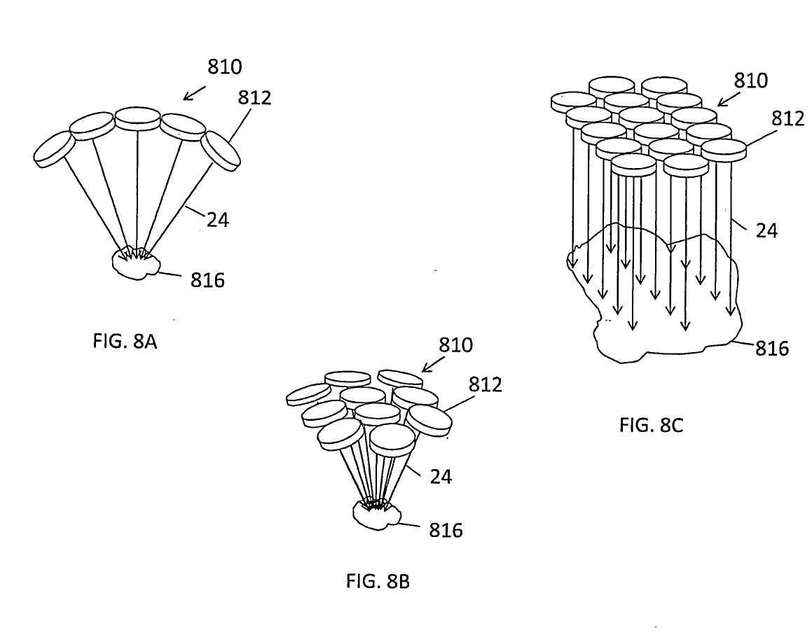

Shown in FIGS. 8A-8C are exemplary geometrical arrangements of the sparkers of the array 810. Each array configuration may deliver a different spatial profile of sound or light output in the region 816. The array may be configured to deliver a maximal acoustic or light output to region 816. At least one sparker 812 of array 810 in FIGS. 8A-8C may include a reflector (not shown), such as reflector 514 described with reference to FIG. 5. Alternatively or in addition, each array 810 of FIGS. 8A-8C may also include a reflector (not shown) associated with at least two sparkers, such as reflector 714 of FIG. 7. For simplicity, the sound emitted from each sparker 812 is illustrated as a single and direct shock wave 24 from each sparker 812 to region 816.

As shown in FIG. 8A, array 810 includes a plurality of sparkers 812 located on a concave line with respect to region 816. As shown in FIG. 8B, sparkers 812 of array 810 may be located on a curved surface, such as a section of the surface of a sphere, with respect to region 816. As shown in FIG. 8C, array 810 may include a plurality of sparkers 812 arranged in a plane with respect to region 816, which may be a planar surface. The planar configuration shown in FIG. 8C may be particularly suited for meat tenderization, where the meat can be located at region 816. For instance, meat products can move along through region 816 under the array, which may be pulsing, so as to provide the right amount of pressure to accomplish tenderization by the time the meat products leave region 816.

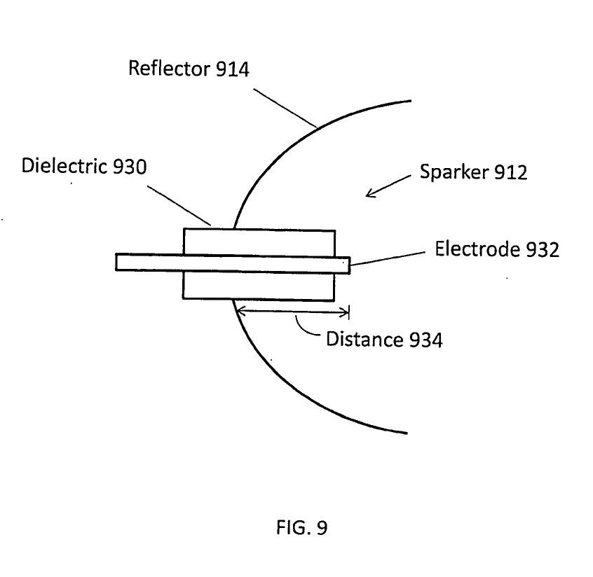

Shown in FIG. 9 is a sparker head design for sparker 912 in which a dielectric material 930 separates and insulates the sparker electrode 932 from surrounding electrically conducting material of the sparker source, such as metallic reflector 914. The tip of electrode 932 is exposed to the environment, for example, a coupling liquid. The separation distance 934 between the tip of electrode 932 and the metallic reflector 914 can be adjusted to control the path of discharge of the sparker 912. The distance 934 may be set large enough to avoid discharge to metallic reflector 914.

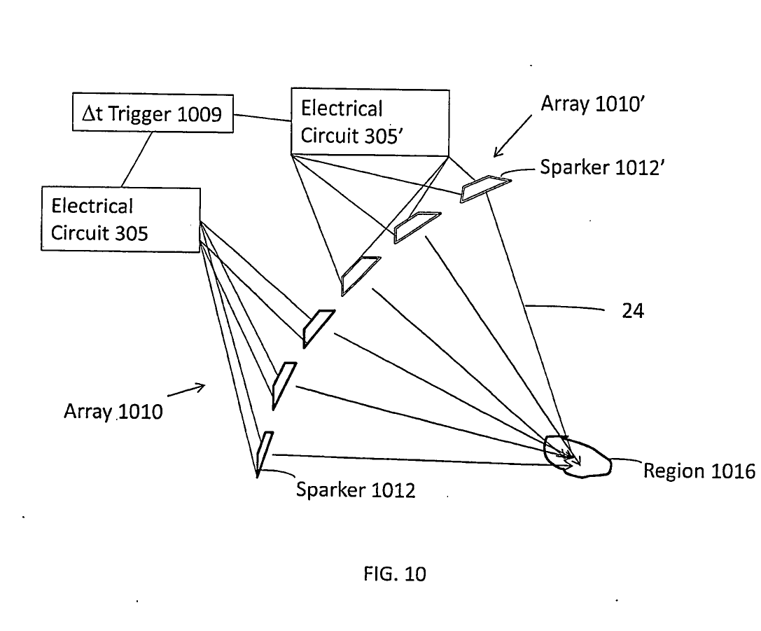

Shown in FIG. 10 are two sparker arrays 1010 and 1010′, each with a separate electrical driver circuit 305 and 305′. Each circuit 305 and 305′ is triggered with a specified time delay 1009, so that two pressure pulses are delivered to region 1016. The two pressure pulses may be maximal pressure pulses spaced in time. One pulse, e.g., the second pulse, may be smaller than the other. Each sparker array 1010 and 1010′ may include one or more reflectors (not shown), such as reflectors 414 described with reference to FIG. 4. In a preferred embodiment, each array includes ellipsoid reflectors, such as reflectors 614 described with reference to FIG. 6. Furthermore, sparkers 1012 and may be located at first focus of the reflectors and the second focus of all the reflectors may be located in the same region. For simplicity, the sound emitted from each sparker 1012, 1012′ is illustrated as a single and direct shock wave 24 from each sparker 1012, 1012′ to region 1016.

In lithotripsy, the use of two sparker arrays delivering two pulses separated by a time delay may accelerate breaking up of the kidney stone. A small interpulse interval, e.g., between 1 and 1000 microseconds, may be used to accelerate breaking up of the stone. In addition, the second pulse may be smaller than the first pulse, or vice versa, which may reduce the risk of tissue damage, yet the double pulse may still accelerate kidney stone breakup when compared to conventional single pulse techniques.

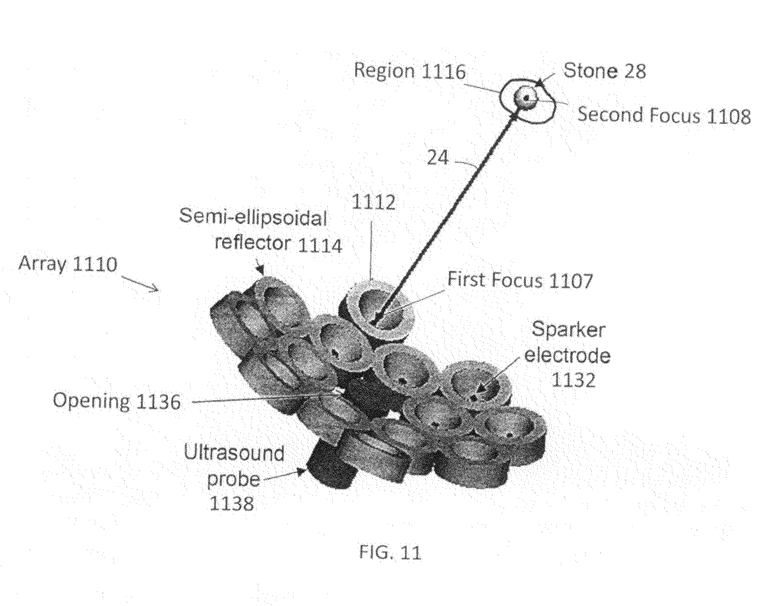

Shown in FIG. 11 is an embodiment of an array 1110 of sparkers sources 1112, including reflectors 1114, configured to provide an opening 1136, which allows a probe 1138 to interrogate and/or observe region 3. Region 1116 encompasses a target, such as kidney stone 28. Probe 1138 may be an ultrasound probe and may be located in-line with focal region 1116. Such an arrangement of sparkers 1112 and probe 1138 can allow for continuous monitoring the position of a kidney stone during a lithotripsy procedure. Consequently, movement of the stone from the shock or from breathing by the patient can be detected and the delivery of shocks adjusted in order to avoid shocks that may miss or only partially hit the stone. Adjustment of the delivery of shocks may include adjustment of the timing, amplitude, or temporal pulselength of the shocks, or a combination of thereof. In general, the sparker array may be positioned at the start of the lithotripsy procedure so that the kidney stone is at a focus of one or more reflectors of the sparker sources. During the procedure, the position of the sparker array may be adjusted in response to a detected position or detected movement of the kidney stone.

As shown in FIG. 11, reflectors 1114 can be semi-ellipsoidal reflectors. Each sparker 1112 can include one electrode 1132. Sparkers 1112 can be located at the first focus 1107 of semi-ellipsoidal reflectors 1114. As shown in FIG. 11, the electrode 1132 of each sparker 1112 can be located at the first focus 1107. The second focus 1108 of all the reflectors 1114 may be located in the same region 1116. For simplicity, the sound emitted from each sparker 1112 is illustrated as a single, direct shock wave 24 from each sparker 1112 to region 1116.

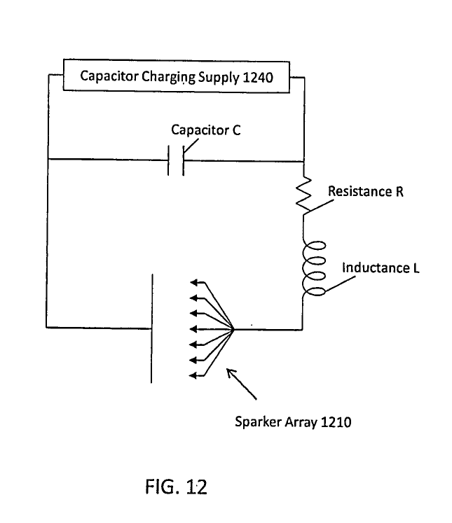

FIG. 12 shows a general circuit configuration for the pulsed electrical driver for the sparker array, such as for circuit 305 that drives array 310 of FIG. 3. A power supply 1240 charges a capacitor C to a specific voltage, and the circuit is characterized by an inductance L and resistance R, as is known in the art. The sparkers in the array 1210 are connected in parallel, each having an effective resistance, inductance and capacitance (not shown).

The temporal pulselength of a pressure pulse emitted from a sparker source can be increased or decreased by increasing or decreasing, respectively, the temporal pulselength of the electrical discharge produced by the electrical circuit driving the sparker source. For arrangements such as shown in FIG. 11, this adjustment of the temporal pulselength of one or more sparkers in the array can produce an adjustment in the size of the focal region at the second focus. Increasing the size of the region at the second focus may increase the capability of the pulse to break up a kidney stone. In single and multiple array systems, the pulselength can vary independently of the pulse intensity.

The way by which adjusting the temporal pulselength can adjust the pressure profile delivered to a region can be understood by considering an instructive example of two pulses. In this example, increasing the pulselength of two pulses also increases the size of the focal region. At the focus, two pulses (with the same peak pressure) arrive simultaneously and combine to produce a peak pressure double that of a single pulse. For locations away from focus, the path length of the two pulses is different, so that the peak pressure is less than double that of a single pulse. The boundary (and hence size) of the focal region is often specified as the location where the peak pressure has fallen to one-half the peak pressure at focus. For two pulses, this occurs when the two pulses no longer overlap. This occurs when the difference in path length (Lp) of the two pulses is equal to the product of tp and c, where tp is the temporal pulselength and c is the propagation speed of a pulse. Consequently, doubling the pulselength tp also doubles the size of the path length which defines the size of the focal region. Thus, the size of the focal region is doubled. Note that the path length difference is related to but does not necessarily coincide with the spatial width of a pulse.

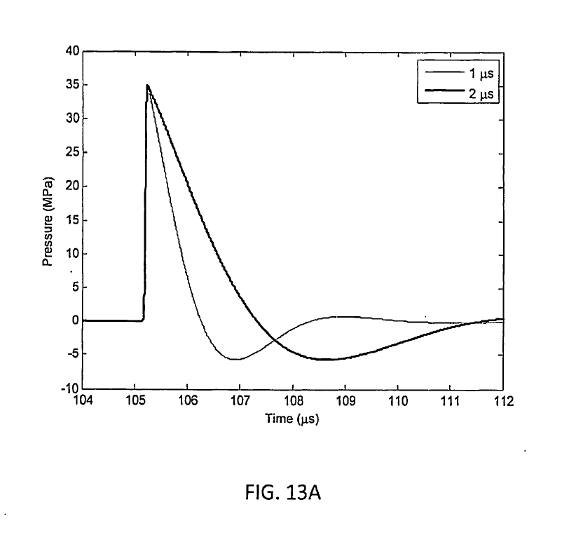

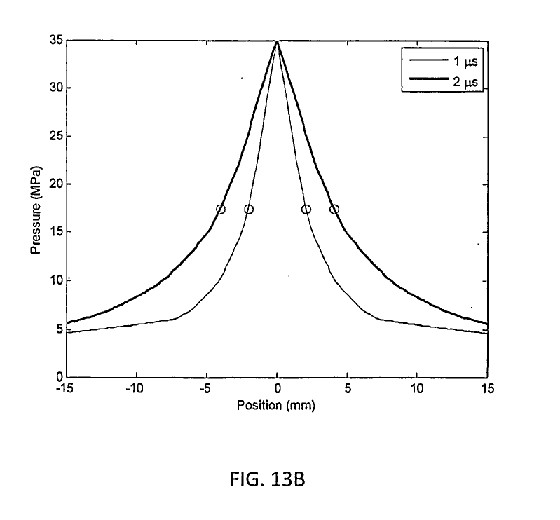

FIGS. 13A and 13B show exemplary simulation data from a model of a sparker array demonstrating that doubling the pulselength also doubles the spatial width of the pulse. FIG. 13A shows a plot of the pressure at the focus for a 1 microsecond pulse (thin line) and a 2 microsecond pulse (thick line), each pulse delivered to the focal region from an array of fourteen sparkers. Pressure in megapascal (MPa) is shown in the vertical axis and time in microseconds (vs) is shown on the horizontal axis. FIG. 13B shows the spatial pressure profiles at the focal region corresponding to the pressure pulses of FIG. 13A. Pressure in megapascal (MPa) is shown on the vertical axis and position in millimeters (mm) is shown on the horizontal axis. The focus is located at the 0 mm position. The boundary for each of the pressure profiles, and hence the size of the focal region, is indicated by circles that mark the position away from the focus at which the pressure is at one-half peak pressure. As shown in FIG. 13B, the size of the focal region changes from 4 mm to 8 mm when the pulselength is doubled from 1 to 2 microseconds. For the simulation data shown, the pulse propagation speed c is 1.5×10⁵ cm/sec.

While this invention has been particularly shown and described with references to preferred embodiments thereof, it will be understood by those skilled in the art that various changes in form and details may be made therein without departing from the scope of the invention encompassed by the appended claims.

What is claimed is:

1. A sparker array comprising:
a plurality of sparker sources of sound and light emissions, the plurality of sparker sources arranged in a geometric pattern with respect to a region,
the array configured to deliver a maximal acoustic or light output to the region.

2. The sparker array of claim 1, wherein at least one sparker source includes a reflector.

3. The sparker array of claim 1, wherein the sparker array further comprises a reflector associated with at least two sparker sources.

4. The sparker array of claim 1, wherein the array configuration is chosen to deliver a specific pressure profile in the region.

5. The sparker array of claim 1, wherein the array is driven by a single electrical circuit.

6. The sparker array of claim 5, wherein the circuit comprises a capacitance of between 0.002 and 128 microfarads and a charge voltage between 3 and 32 kilovolts.

7. The sparker array of claim 1, wherein the sound and light emissions occur in a liquid.

8. The sparker array of claim 7, wherein the salinity of the liquid is greater than 1 millisiemens per centimeter.

9. The sparker array of claim 1, where in the number of sparker sources in the array is selected to optimize the pressure output from the array to the region.

10. The sparker array of claim 1, wherein each sparker source includes one electrode.

11. The sparker of claim 10, wherein the electrode of each sparker source is insulated from other electrically conducting material of the sparker source.

12. The sparker array of claim 10, wherein at least one sparker source further includes a semi-ellipsoidal reflector having a focus, the electrode of said at least one sparker source being located at the focus.

13. The sparker array of claim 1, wherein the array is configured to provide an opening to allow a probe to interrogate the region.

14. The sparker array of claim 13, wherein at least one sparker source further includes a reflector having a first focus in the vicinity of the reflector and a second focus in the region, the at least one sparker source being located at the first focus and the probe being aligned with the second focus.

15. The sparker array of claim 1, wherein the sound and light emissions include pulses having an adjustable temporal pulselength.

16. The sparker array of claim 15, wherein the pulselength is adjusted to adjust the pressure profile delivered to the region.

17. A sparker system comprising:
two or more sparker arrays, each sparker array comprising:
a plurality of sparker sources of sound and light emissions, the plurality of sparker sources arranged in a geometric pattern with respect to a region,
each array configured to deliver a maximal acoustic or light output to the region.

18. The system of claim 17, wherein each sparker array is driven by an electrical circuit.

19. The system of claim 18, wherein the electrical circuits of the sparker arrays are triggered with a time delay.

20. The system of claim 19, wherein the delay is between 1 and 1000 microseconds.

21. The system of claim 17, wherein at least one sparker source includes a reflector.

22. The system of claim 17, wherein the sound and light emissions occur in a liquid.

23. A method of delivering sound or light to a region comprising:
providing a first array of sparker sources of sound and light emissions, the sparker sources arranged in a geometric pattern with respect to the region; and
delivering a first acoustic or light output to the region using the first array.

24. The method of claim 23, wherein at least one sparker source includes a reflector.

25. The method of claim 23, wherein delivering comprises driving the first array with a single electrical circuit.

26. The method of claim 25, further comprising selecting the number of sparker sources in the first array to optimize the pressure output from the first array to the region.

27. The method of claim 23, further comprising observing the region using a probe.

28. The method of claim 23, wherein the sound and light emissions include pulses having an adjustable temporal pulselength.

29. The method of claim 28, further comprising adjusting the pulselength to adjust the pressure profile delivered to the region.

30. The method of claim 23, further comprising providing a second array of sparker sources of sound and light emissions, the sparker sources of the second array arranged in a geometric pattern with respect to the region, and delivering a second acoustic or light output to the region using the second array.

31. The method of claim 30, wherein delivery of the second acoustic or light output is triggered with a time delay after delivery of the first acoustic or light output is triggered.
